(19) United States
(12) Patent Application Publication
(10) Pub. No.: US 2010/0215707 A1
(43) Pub. Date: Aug. 26, 2010

(54) ACTIVATED CREATININE AND PRECURSORS THEREOF AS ANTIBACTERIAL AGENTS, COMPOSITIONS AND PRODUCTS CONTAINING SUCH AGENTS AND USES THEREOF

(76) Inventors: Thomas McDonald, Omaha, NE (US); Steven Tracy, Omaha, NE (US); Annika Weber, Omaha, NE (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/711,727

(22) Filed: Feb. 24, 2010

Related U.S. Application Data
(60) Provisional application No. 61/208,488, filed on Feb. 25, 2009.

(51) Int. Cl. A61K 31/4168; A61K 31/221; A61K 9/12; A61K 9/70; A61P 17/02

(52) U.S. Cl. 424/409; 514/386; 514/551; 424/46

(57) ABSTRACT

Creatinine, creatinine precursors or the pharmaceutically acceptable salts thereof are activated to function as an antibacterial agent which has broad spectrum activity and is beneficially used in a variety of applications, such as antimicrobial wound dressings, compositions for topical delivery of the antibacterial agent and for preventing and/or inhibiting the occurrence or spread of bacterial infection, as well as the growth of odor-causing bacteria, to name a few.

### CROSS-REFERENCE TO RELATED APPLICATIONS

The present application claims the benefit of U.S. Provisional Patent Application No. 61/208,488, filed Feb. 25, 2009, the entire disclosure of which is incorporated by reference herein.

### FIELD OF THE INVENTION

The present invention relates generally to antibacterial agents, products incorporating such agents and the use thereof in preventing the occurrence and spread of bacterial infection, as well as treating certain bacteria-mediated dermatological conditions. More specifically, the present invention provides an antibacterial agent, which is derivable from a natural source and which has a broad spectrum of activity in topical applications, including activity against bacteria of known antibiotic resistance, e.g., methicillin-resistant Staphylococcus aureus (MRSA).

### BACKGROUND OF THE INVENTION

Numerous bacterial strains that are resistant to the most commonly-used antibiotics have been widely reported in recent years. Methicillin-resistant Staphylococcus aureus (MRSA) and other highly-resistant strains are now fairly commonplace, posing ever more severe threats to human health.

Wound dressings with built-in antimicrobial protection are used in hospitals to help reduce the incidence of nosocomial infection. Those currently on the market deliver to the wound site agents such as silver, polyhexamethylene biguanide (PHMB), chlorhexidine, 5-chloro-2-(2,4-dichlorophenoxy)phenol(Triclosan) and the like. It has been reported, however, that silver-containing antimicrobial wound dressings delay wound healing and may be toxic to cells involved in the healing process, including both keratinocytes and fibroblasts. www.worldwidewounds.com/2004/february/Cooper/Topical-Antimicrobial-Agents.html; www.ncbi.nih.gov/pubmed/15019121. Moreover, prolonged exposure to silver is known to produce a bluish-gray discoloration of the skin, deep tissue, nails and gums, known as argyria, for which there is no known treatment. Exposure to silver can also cause neurological problems, e.g., seizures, as well as allergies in atopic individuals.

Contact with PHMB has been reported to induce anaphylaxis and erythema multiforme in certain individuals. Schweiz. Med. Wochenschr., 128: 1508-11 (1998); Jehanno et al., Int. J. Occup. Med. Envirn. Health, 19(1): 81-2 (2006). Anaphylaxis has likewise been experienced with the use of chlorohexidine. R. Jee; Br. J. Anesth., 103(4): 614-15 (2007). R. Evans, BMJ, 304(6828): 686 (1992). There is also evidence that Triclosan, a chlorophenol derivative, can cause photoallergic contact dermatitis, which occurs when skin exposed to Triclosan is also exposed to sunlight. www.lindachae.com/Trichlosan_article.htm. Alternative wound care products that deliver antibacterial agents comprising an organic substance that naturally occurs in mammals would afford notable advantages over products based on elemental metals or synthetic agents, especially in long-term wound treatment.

While the search for new and effective antibiotics and antibacterials is ongoing, success has been elusive in many instances due to the capacity with which bacteria tend to become resistant to such agents over time through mutation and/or gene exchange. C. Walsh, Nature Reviews, 1: 65-70 (2003); C. Walsh, Nature, 406: 775-781 (2000). Indeed, there is growing public health concern over the appearance of bacteria which are increasingly resistant to both first-line and last resort antibiotics, and for which there is a dearth of effective broad-spectrum treatments.

Properties of an ideal antibacterial agent would be one that is (i) not susceptible to genetic bypass, (ii) safe even at high concentrations, (iii) stable, and (iv) capable of suppressing the replication of and/or killing both gram negative and gram positive bacteria. There is a pressing need for antibacterial agents that satisfy these criteria.

Creatinine (2-amino-1-methyl-4-imidazolidinone) is a stable, natural end-product of creatine catabolism in muscle tissue. It is present in serum and in urine at approximately 100 μM concentrations.

Creatinine at 8.8 mM has previously been used to support the growth of a strain of Pseudomonas aeruginosa, P. Kopper, J. Bacteriol., 54: 359-62 (1947). U.S. Pat. No. 4,275,164 discloses a creatinine-containing nutrient medium for growing an aerobic soil microorganism from which a creatinine iminohydrolase enzyme preparation is obtainable. Creatinine has also been shown to inhibit arginine deiminase (3.5.3.6.) in Streptococcus faecalis (since reclassified as Enterococcus faecalis). B. Petrack et al., Arch Biochem Biophys., 69: 186-197 (1957).

Insofar as is known, it has not previously been reported that creatinine or its precursors could be used safely and effectively as a broad spectrum antibacterial agent in place of, or in combination with existing antibiotics and antibacterials.

### SUMMARY OF THE INVENTION

In one embodiment of the present invention, there is provided an antibacterial composition comprising, as the active agent, antibacterially-activated creatinine, a pharmaceutically acceptable salt of antibacterially-activated creatinine, a precursor of antibacterially-activated creatinine, a pharmaceutically acceptable salt of such precursor, or a combination thereof, and a suitable carrier medium.

According to another embodiment of this invention, there is provided a wound dressing comprising a wound dressing material in which is incorporated an antibacterially effective amount of at least one of antibacterially-activated creatinine, a pharmaceutically acceptable salt of antibacterially-activated creatinine, a precursor of antibacterially-activated creatinine or a pharmaceutically acceptable salt of such precursor.

Antibacterially-activated creatinine, creatinine precursors and pharmaceutically acceptable salts thereof, as described herein, can also be incorporated into conventional wound treatment preparations, to improve the efficacy thereof.

According to yet another embodiment, the present invention provides fibrous articles which comprise at least one of antibacterially-activated creatinine, a pharmaceutically acceptable salt thereof, a precursor of antibacterially-activated creatinine and a pharmaceutically acceptable salt of such precursor incorporated as an antibacterial agent in the fibrous article, in an amount effective to impart antibacterial properties to the article. Fibrous articles that can be rendered resistant to bacterial colonization in accordance with this invention include, without limitation, natural and synthetic fibers, woven or non-woven fabric, paper, cardboard, pressed wood or fiber board.

In still another embodiment, the present invention provides personal care products comprising the above-described antibacterial agent and a dermatologically acceptable carrier medium.

Regarding the uses of the above-mentioned antibacterial agents, the present invention provides a general method of inhibiting growth (propagation) of bacteria by administration of such agents to a surface area in need of bacterial growth inhibition. The method may be practiced on either humans or non-human animal subjects or on inanimate objects. More particularly, the method can be performed to treat or prevent infection in a wound and/or inhibit bacterial colonization of a wound site by applying an antibacterial agent of the invention directly to the wound site, or by first putting the antibacterial onto a wound dressing, which is then applied to the wound site.

In other embodiments, the antibacterial agents of the invention can be used effectively to suppress or prevent body odor by inhibiting the growth of odor-causing bacteria, as well as to treat or prevent bacterial colonization of body orifices.

In a further embodiment of the invention, a method is provided for rendering substrates resistant to bacterial colonization by including therein the above-described antibacterial agents. Examples of substrates that can be made to resist bacterial colonization in this way include, without limitation, fibers, film and sheet materials of various thickness, as well as coated or molded substrates.

Therapeutic treatment methods are also included within the scope of this invention. Specifically, a method is provided for treating bacterial-mediated dermatologic conditions by administering one or more of the antibacterial agents of the invention, together with an effective amount of a therapeutic agent for providing relief from and/or alleviating the symptoms of such conditions.

In yet another embodiment, the present invention provides a method of suppressing bacterial growth in a culture comprising a eukaryotic organism and a growth medium for such organism, by adding to the growth medium an antibacterially effective amount of an antibacterial agent of this invention. Such a method may be advantageously used to promote selective propagation of commercially important fungal, yeast or eukaryotic cells, while inhibiting undesirable bacterial growth.

As the following detailed description of the invention will make clear, antibacterially-activated creatinine and creatinine precursors are highly effective in suppressing replication of diverse gram negative and positive bacteria, including MRSA, Vancomycin-resistant Enterococci (VRE) and high level resistance bacterial strains.

### BRIEF DESCRIPTION OF THE DRAWINGS

FIG. 1 is a graphical representation showing the relative activity of antibacterially-activated creatinine after acid activation using various acid treatments;

FIG. 2 includes graphical representations of the effects of the antibacterial agent of the invention on the growth of S. aureus and Micrococcus luteus. In FIG. 2A a precursor of antibacterially-activated creatinine, i.e., creatine ethyl ester (CEE), is the antibacterial agent and growth of S. aureus is represented by absorbance (A₅₈₀) readings plotted as a function of time (hrs), whereas in FIG. 2B, CEE is the active agent and growth of M. luteus is represented by absorbance (A₅₄₀) readings plotted as a function of time (hrs);

FIG. 3 is a graphical representation of the growth curves of three (3) bacterial species (S. aureus, M. luteus and Escherichia coli) in Lederberg's broth (LB), with the addition of antibacterially-activated creatinine (CRN) to 100 mM in early log phase of growth and absorbance readings plotted as a function of time (hrs);

FIG. 4 is a graphical representation of data showing that, when added to bacterial culture to a final concentration of 200 mM, a precursor of antibacterially-activated CRN, i.e., CEE, and antibacterially-activated CRN, are bactericidal for S. aureus, starting at approximately 1×10³ organisms per mL;

FIG. 5 is a graphical representation of the relative activities of various antibacterially-activated creatinine precursors, namely, the ethyl, propyl, octyl and benzyl esters of creatine;

FIG. 6 is a graphical representation of the antibacterial activity of a precursor of antibacterially-activated CRN, i.e., CEE, showing that formulations including commercial, water-based lotion or cream carrier media were effective to inhibit growth of S. aureus. Anhydrous formulations of a precursor of antibacterially-activated CRN, CEE, and antibacterially-activated CRN were included as a basis of comparison; Anhydrous formulations of a precursor of antibacterially-activated CRN, CEE, and antibacterially-activated CRN were included as a basis of comparison;

FIG. 7 is a graphical representation of the antibacterial activity of antibacterially-activated CRN and antibacterially-activated CEE in a hydrogel carrier;

FIG. 8 is a graphical representation showing the effects of different concentrations of antibacterially-activated CRN incorporated into a fabric substrate, serving as a surrogate wound dressing, to which bacteria were applied and suspended in growth media; in FIG. 8A the antibacterial activity of CRN-treated fabric was compared to a culture control (CC) over time; in FIG. 8B the antibacterial activity of three different concentrations of antibacterially-activated CRN were compared to an untreated control;

FIG. 9 is a graphical representation of the antibacterial activity of a commercial bandage pre-treated with a precursor of antibacterially-activated CRN, CEE;

FIG. 10 shows the inhibitory effect of antibacterially-activated CRN on growth of two (2) major body odor-producing organisms in culture; in FIG. 10A, the test organism is Brevibacterium linens (ATCC 9175), whereas in FIG. 10B, the organism is M. luteus skin isolate; and

FIG. 11 shows the results of tests using a precursor of antibacterially-activated CRN, i.e., CEE, as a media supplement to determine its capability to suppress undesired bacterial growth in culture comprising a eukaryotic organism and a growth medium for such organism. In FIG. 11A, the antibacterial agent is either absent or included at 200 mM in the culture media and exhibits selectivity for the yeast Saccharomyces sp. grown in the presence of Micrococcus sp.; in FIG. 11B, the antibacterial agent is included at various concentrations in the culture media and exhibits selectivity for the yeast Rhodotorula sp. grown in the presence of S. aureus; in FIG. 11C the antibacterial agent is included at two (2) different concentrations and tested on three different organisms, one (1) bacterium and two (2) yeast sp.

### DETAILED DESCRIPTION OF THE INVENTION

The present inventors have discovered that, by appropriate treatment, creatinine, creatinine precursors and pharmaceutically acceptable salts thereof can be caused to function as effective antibacterial agents. The treatment process, referred to herein as “antibacterial-activation” brings about chemical modification that imparts broad spectrum antibacterial activity to the creatinine molecule Additionally, activation may include physical or structural changes in the creatinine molecule that are necessary for generating antibacterial activity.

Experiments conducted to date indicate that antibacterial activation of creatinine requires pH adjustment of the surrounding medium to below 6.5, and preferably between 5.0-5.5. However, all of the factors that influence antibacterial activity of creatinine or its precursors have not been definitively determined. The data show that acquisition of antibacterial activity is not merely a matter of maintaining a pre-determined pH, given that chemically distinct species of activated creatinine exhibit different levels of activity at essentially the same pH. Additionally, adjusting the pH of media or carrier to 5.0-5.5 in the absence of CRN does not generate antibacterial activity. It appears that the observed differences in the degree of antibacterial activity may be accounted for, at least in part, by the nature of the counter-ion associated with the activated creatinine. As exemplified below, creatinine activated with acetic acid has substantially greater antibacterial activity than creatinine activated with nitric or hydrochloric acid. However, there is insufficient data at hand to conclude whether, as a general proposition, organic acids are superior to inorganic acids as creatinine activators.

The terms “antibacterially-activated” or “antibacterial-activation”, as used herein, refer to the conversion of creatinine, creatinine precursors or pharmaceutically acceptable salts thereof from a state in which such chemical species have no appreciable antibacterial activity to one in which they exhibit an antibacterial effect.

The term “antibacterial”, as used herein to characterize the agents, compositions, products and methods of this invention, refers to the property of the antibacterially-activated creatinine, creatinine precursor and pharmaceutically acceptable salts thereof by which the propagation of bacteria is inhibited (bacteriostatic property), or bacteria are killed (bactericidal property).

The creatinine used in the practice of this invention can be isolated from natural sources, e.g., urine, or prepared by treating commercial creatine with mineral acids, E. Hinegardner, J. Biol. Chem., 56: 881 (1923), and is illustrated as follows:

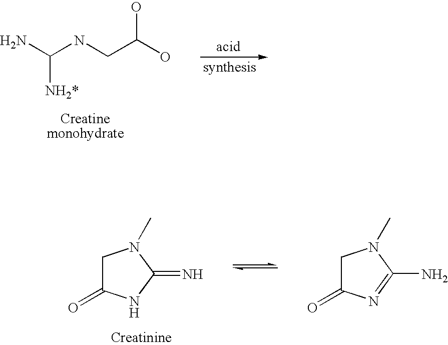

Creatinine is also commercially available, e.g., from Sigma-Aldrich Company.

Antibacterially-activated creatinine can also be derived from a creatinine precursor. The term “creatinine precursor” as used herein refers to any compound that can be caused to undergo conversion to creatinine. Preferred embodiments of creatinine precursors include creatine and its esters, such as the ethyl, propyl, octyl and benzyl esters, and pharmaceutically acceptable salts thereof. These esters can be prepared in the manner described in U.S. Pat. No. 6,897,334 to Vennerstrom. See also A. Dox, J. Biol. Chem., 54: 671-73 (1922). Creatine ethyl ester is known to undergo non-enzymatic cyclization to form creatinine. A. Giese and C. Lecher, Biochem. Biophys. Res. Commun., 388: 252-55 (2009).

Creatine esters can be synthesized following procedures which are familiar to those skilled in the art. The synthesis of the ethanol ester and its conversion to CRN is illustrated as follows:

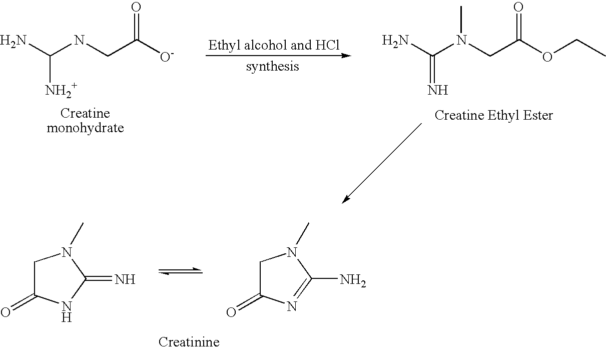

The antibacterial agents of the invention may be used in the form of a pharmaceutically acceptable salt. As used herein, the term “pharmaceutically acceptable”, such as in the context of “pharmaceutically acceptable salt”, refers to a compound that is not biologically or otherwise undesirable, i.e., the compound may be incorporated into a carrier medium and administered to a subject without causing any undesirable biological effects or interacting in a deleterious way with any of the other ingredients of the composition with which it is combined. The antibacterial agents of the present invention form pharmaceutically acceptable salts with various acids including, without limitation, hydrochloric acid, malic acid, nitric acid, phosphoric acid, citric acid and acetic acid. These salts can be prepared following procedures which are familiar to those skilled in the art.

The antibacterial composition of the present invention comprises one or more of the antibacterial agents described above in a suitable carrier medium. The particular carrier medium selected for preparation of the composition will be determined by its end use. That is to say, an antibacterial personal care product will ordinarily include a different carrier from an antibacterial composition that is incorporated into a garment or a dust cloth, for example. In the case of personal care products, a dermatologically acceptable carrier is used. The term “dermatologically acceptable carrier” refers to a carrier medium or vehicle suitable for topical application to a body surface, including skin or mucosal tissue. The carrier medium may be aqueous or anhydrous (non-aqueous), and in liquid or solid form. The term “solid” as used herein also includes semi-solid substances. Representative examples of suitable aqueous liquid carriers include, without limitation, water, water-containing solutions, e.g., hydroalcohols, and other forms of carrier media described hereinbelow. The term “aqueous” as used herein refers to a material or composition that comprises water as a component at the time of its preparation or formulation, or thereafter becomes infused with water in the environment of use. Representative examples of non-aqueous liquid carriers include, without limitation, mineral oil, polyethylene glycol, vegetable oil, fatty acids, propylene glycol, glycerin, alcohol, paraffin, or a mixture thereof.

In certain applications involving localized delivery of the antibacterial agent to a site of subcutaneous bacterial infection, an injectable carrier medium is used.

Topical compositions comprising the antibacterial agents of this invention may be in any form suitable for application to a body surface including, for example, ointment, cream, gel, lotion and paste forms, which may be formulated as an occlusive or semi-occlusive composition to provide enhanced hydration. Ointments are semi-solid preparations normally having a petrolatum (soft paraffin) or other petroleum derivative base, which is classified as either an oleaginous, emulsifiable, emulsion or water-soluble base. Creams are viscous liquids or semi-solid emulsions, which may be oil-in-water or water-in-oil emulsions. Gels are semi-solid suspension systems that comprise an organic macromolecule distributed substantially uniformly throughout a liquid carrier medium, which is normally aqueous, but may also contain an alcohol and, optionally, an oil. Lotions are usually liquid or semi-liquid preparations in which solid particles are present in a water or alcohol base. Pastes are semi-solid carrier vehicles in which an active ingredient is suspended in a suitable base material, such as petrolatum, hydrophilic petrolatum or the like, which form a fatty paste. A paste may also be prepared from a single-phase aqueous gel of the type described above, using carboxymethyl cellulose or the like as a base material.

L. V. Allen, The Art, Science and Technology of Pharmaceutical Compounding, 2^{nd} Ed., Chapter 18, Ointments, Creams and Pastes provides additional detailed information pertaining to carrier media which may be used to formulate the antibacterial compositions of this invention. Except insofar as any conventional carrier medium or vehicle is incompatible with the antibacterial agents of the invention, such as by producing any undesirable biological effect or otherwise deleteriously affecting any other component of the antibacterial composition, its use is contemplated to be within the scope of this invention.

In the antibacterial compositions of the invention, the antibacterial agent may be present in an amount of at least 0.5% and preferably from about 3% to about 99.5%, such percentages being based on the total weight of the composition. When used in an aqueous form, the antibacterial agent may be present in an amount of at least 10 mM and preferably from 100 mM to 2M. Anhydrous forms of the antibacterial compositions of the invention may include the antibacterial agent in an amount of at least 2%, and preferably from 10%-40% based on the total weight of the composition.

The composition may include both antibacterially-activated creatinine and a precursor thereof to afford longer lasting antibacterial action than would be obtainable with the antibacterially-activated creatinine alone. The presence of antibacterially-activated creatinine provides initial antibacterial activity while the creatinine precursor(s), such as creatine ethyl ester, is converted to antibacterially-activated creatinine only when they come in contact with water or water-containing substances. Therefore the antibacterial activity of the composition is prolonged, due to the gradual conversion of creatinine precursor to the antibacterially-activated creatinine over time.

One or more supplemental active agents may also be incorporated in the antibacterial composition of the invention. For example, an anti-infective agent may be advantageously used in combination with the antibacterial agent described herein. Such anti-infective agents include, without limitation, antibiotic, anti-fungal, antiseptic and anti-viral agents. As specific examples, there may be mentioned penicillins, macrolides, cephalasporins, polypeptides, polyenes, imidazoles, triazoles, alcohols, boric acid, iodine and silver.

The antibacterial compositions of the invention may also comprise one or more optional ingredient known in the art, such as diluents, viscosity modifiers, surfactants, preservatives, coloring agents, perfumes, humectants, emollients, skin penetrating enhancers, emulsifiers, suspension or dispersion aids, stabilizers, buffers, UV absorbers/sunscreens, an aerosol propellant, or combinations thereof. Numerous examples of such ingredients are set forth in U.S. Patent Application Publication No. US 2005/0232957.

The above-described antibacterial agents and compositions may be incorporated into wound dressings for applications in which antimicrobial wound dressings are currently utilized. See, for example, U.S. Pat. Nos. 6,168,800, 5,833,665 and 5,738,861 and U.S. Patent Application Publication No. 2004/0001880. These include sterile field applications, such as surgery and central venous line placement and care, and in aseptic techniques, such as wound care, peripheral IV catheter insertion and care, or the like. Other embodiments include field dressings of the type found in a military first aid case, and adhesive plastic and fabric film bandages, e.g., Band-Aid™-type bandages.

As previously noted, the wound dressing embodiment of the invention comprises a wound dressing material in which is incorporated an antibacterially effective amount of at least one antibacterially-activated creatinine, a pharmaceutically acceptable salt thereof, a precursor of antibacterially-activated creatinine, or a pharmaceutically acceptable salt of the precursor. The wound dressing material may be selected from the group of a hydrocolloid, a hydrogel, a semi-permeable transparent film, an open-cell foam, an alginate, an absorptive filler, a woven fabric and a non-woven fabric or a combination of such materials.

The selection of a particular wound dressing is normally made on the basis of functionality (absorption of wound exudates, control of bleeding or fluid loss, maintenance of moist wound surface and protection against contamination, desiccation and abrasion), wound size and avoidance of trauma upon removal from the wound site.

Hydrocolloidal wound dressing material typically comprises an absorbent and elastomer dispersed in an adhesive base. Carboxymethylcellulose is commonly used as the absorbent component. Some hydrocolloid dressings contain pectin. These dressings are moisture retentive and promote autolytic debriding. They are also highly occlusive, providing protection against exogenous contaminants. They are available in wafer form in a variety of shapes, as well as granules, powders and paste. Representative examples of dressings of this type include Comfeel, Duo Derm and Repli Care. See also, U.S. Pat. Nos. 6,033,684, 4,551,490 and 4,393,080. Hydrocolloid dressings may be secured to a wound site by means of a transparent film cover which is impermeable to liquid, bacteria and viruses. Alternatively, the hydrocolloid wound dressing material may be laminated to a backing film.

A hydrogel can be described generally as an insoluble polymer with hydrophilic sites which absorb and interact with significant volumes of liquid, particularly water or in the case of wound dressings, wound exudates. A hydrogel-based wound dressing material typically comprises cross-linked hydrophilic macromolecules containing up to about 95% water by weight. These dressings are effective for establishing and maintaining a moist microenvironment for cell migration and rehydrating eschar and slough for easy removal from the wound. They also diminish wound pain. Representative examples of hydrogel dressings include, without limitation, Solo Site, Intra Site and Carrasyn Gel. See also, U.S. Pat. Nos. 6,238,691, 5,112,618, 5,106,629 and 4,909,244. The hydrogel material may be in sheet or gel form, and in the latter case can be applied directly to the wound, or impregnated in an absorbent compress, e.g., gauze, which is used for dressing the wound. The absorbent compress may be bound to the wound by a suitable bandage material.

Alginate wound dressings comprise non-woven fibers of soluble salts of alginic acid, a derivative of seaweed. These dressings are moisture-retentive, non-occlusive and non-adherent, and are capable of absorbing moderate to heavy wound exudates in superficial and deep wounds. They are available in pad (felt) and rope form, the latter being useful as a filler for deep or tunneling wounds. Representative examples of such dressings include, without limitation, Kaltostat™ and Curasorb®. See also U.S. Pat. Nos. 5,836,970, 5,197,945, 4,948,575 and U.S. Patent Application Publication No. 2005/0287193.

In another embodiment, the wound dressing may be in the form of a bandage strip and an absorbent compress attached to the bandage strip. This form of dressing is commonly referred to as a first aid field dressing. Preferably, the absorbent compress is gauze, e.g., cotton or chemical derivative of cellulose, or an open cell foam material (e.g., hydrophilic polyurethane foam, optionally gel film or silicon coated). A wound dressing of this type may be applied as a dry dressing or a water dressing, i.e., a dressing that is kept wet with sterilized water or saline solution. It is conventionally packaged in an air-tight container.

The wound dressing may also be embodied in an adhesive bandage comprising a flexible substrate coated with a pressure-sensitive adhesive coating and an absorbent compress affixed to at least part of the adhesive coated substrate, with the absorbent compress having incorporated therein one or more of the above-described antibacterial agents. The flexible substrate may be a plastic or fabric film, which is in the form of a strip, a patch or a spot. The invention may also be incorporated into pre-surgery bandages for use to effectively sterilize the proposed incision site.

The wound dressings described above facilitate wound care by protecting against bacterial colonization within the dressing and bacterial penetration through the dressing. This protective effect is a direct result of the excellent barrier function imparted by the antibacterial agent of the invention.

In addition to their utility in wound dressings, the above-described antibacterial agents can be used to enhance the efficacy of topical wound treatment preparations, such as ointments, creams, gels, lotions, emulsions, pastes, liniments and collodions. For example, the improvement can be realized by incorporating into standard liniment or collodion preparations an antibacterially effective amount of one or more antibacterial agents of the invention.

Antibacterially-activated creatinine, its precursors and pharmaceutically acceptable salts thereof are also effective for imparting antibacterial properties to fibrous articles, including fibers, threads, yarns, woven fabric and non-woven fabric. These fibrous articles may be used for the manufacture of any number of finished goods including, without limitation, an absorbent compress, a bandage, a wound packing material, a garment, bed clothes, a dust cloth, a tampon, a sanitary napkin and a fluid filter. The bacterial resistant woven and non-woven fabrics of the invention can be made into garments such as a surgical gown, foot protectors, a face mask, a head or hair covering, a diaper and gloves. The bacteria-resistant fibrous articles may also be converted into paper, cardboard, pressed wood or fiber board according to methods conventionally used for the manufacture of such products.

The present invention can also be embodied in a wide variety of personal care products that comprise an antibacterially effective amount of at least one of antibacterially-activated creatinine, a pharmaceutical acceptable salt of said antibacterially-activated creatinine, a precursor of antibacterially-activated creatinine or a pharmaceutically acceptable salt of such precursor admixed with a dermatologically acceptable carrier medium. Examples of such products include, without limitation, a skin care product, hand sanitizer, body lotion, feminine care products, foot care products, deodorant and combinations thereof. The products are packaged in containers appropriate to their intended use, e.g., bottles which may include a pump dispenser or a spray nozzle, an aerosol dispenser, a roll-on dispenser and a stick dispenser.

The skin care products may also include an effective amount of a therapeutic agent for the treatment of a bacteria-mediated dermatological condition. Among the conditions which may be treated with the skin care products of the invention are inflammatory dermatoses, such as acne vulgaris, rosacea, atopic dermatitis and other forms of eczema, as well as impetigo and bacterial folliculitis.

The antibacterial agents described above have numerous practical applications in methods for the treatment and/or prevention of bacterial infection, both for human and veterinary use. As used herein, the terms “treatment” or “treating” refer to the capacity of the antibacterial agents of the invention to provide relief from, alleviation or reduction of the severity or frequency of symptoms, or elimination of the underlying cause(s) of bacterial infection and/or colonization, such as inflammation, redness, soreness, swelling or the like, and the improvement or repair of damage resulting from bacterial infection.

The terms “prevention” or “preventing”, as used herein, refer to the capacity of the antibacterial agents of the invention to avert the occurrence of symptoms and/or the underlying cause(s) of bacterial infection and/or colonization.

Thus, the methods of the present invention encompass both prevention of bacterial infection and/or colonization in a susceptible subject and treatment thereof in a clinically symptomatic subject. As used herein, the term “subject” refers to animals, including mammals and preferably humans, livestock and domestic or companion animals. The term “livestock” encompasses cattle, poultry, swine, sheep and horses. For example, the antibacterial agents or compositions of the invention may be administered to dairy cows for the treatment of mastitis, according to procedures well known in the industry.

Antibacterially-activated creatinine, creatinine precursors and pharmaceutically acceptable salts thereof have shown broad spectrum inhibitory activity with respect to organisms such as Staphylococcus aureus, Enterococcus faecalis, Pseudomonas aeruginosa, Pseudomonas fluorescens, Escherichia coli, Acinetobacter baumannii, Brevibacterium linens, Micrococcus luteus, Bacillus subtilis, Bacillus cereus. As exemplified below, these agents exhibit inhibitory activity against antibiotic resistant organisms, including methicillin-resistant S. aureus (MRSA), Acinetobacter baumannii high level resistance, E. coli beta lactamase producer, Pseudomonas aeruginosa high level resistance and VRE, the most common causes of which are E. faecium and E. faecalis.

The aforementioned method may also be practiced by administering the antibacterial agents at a subcutaneous infection site to treat conditions such as a cyst, a carbuncle, a boil, an abscess or a combination thereof.

The therapeutic and/or prophylactic methods of the invention will normally include medical follow-up to determine the antibacterial effect produced by the antibacterial agents described herein, with or without supplemental therapeutic agent(s), in the subject on whom the method is performed.

Initial testing of the antibacterially-activated creatinine described herein in disc diffusion assays has shown it to be more effective than gentamicin at inhibiting the growth of a broad spectrum of bacteria, including drug-resistant organisms, as shown in the following table.

Antibacterial activity of creatine ethyl ester (CEE) & Gentamicin (Gn).

| | | Zone of | |
|---|---|---|---|
| | | Inhibition | |
| | | (mm) | |
| | Class | Gn | CEE |
| GRAM POSITIVE | | | |
| Staphylococcus aureus | laboratory strain 29213 | 24 | 26 |
| Staphylococcus aureus | UMSA-1 isolate | nd* | 24 |
| Staphylococcus aureus | methicillin resistant (MRSA) | 7 | 27 |
| Staphylococcus epidermidis | laboratory strain | nd | 27 |
| Enterococcus faecalis | laboratory strain 29212 | 14 | 19 |
| Enterococcus faecium | vancomycin resistant (VRE) | 8 | 18 |
| Micrococcus luteus | laboratory isolate | nd | 22 |
| Brevibacterium linens | ATCC 9175 | nd | 40 |
| Bacillus subtilis | laboratory strain | nd | 20 |
| Bacillus cereus | laboratory strain | nd | 22 |
| GRAM NEGATIVE | | | |
| Pseudomonas aeruginosa | laboratory strain 27853 | 21 | 27 |
| Pseudomonas aeruginosa | high level resistance (HLR) | 8 | 25 |
| Pseudomonas fluorescens | laboratory strain | nd | 18 |
| Escherichia coli | laboratory strain 35150 | 21 | 15 |
| Escherichia coli | beta lactamase producer | 24 | 19 |
| | (ESBL) | | |
| Acinetobacter baumannii | high level resistance (HLR) | 6 | 16 |
| YEAST | | | |
| Candida albicans | laboratory strain 24433 | 8 | 0 |
| Rhodotorula sp. | laboratory isolate | nd | 0 |
| Saccharomyces sp. | isolate from baker's yeast | nd | 0 |

*nd Not Done

This testing involved the use of 50 mg of an anhydrous topical cream containing the antibacterial agent of the invention in an amount of 28% by weight and 10 μg of gentamicin, impregnated into a standard commercially available disc (Remel, Lenoxa KN).

The embodiments of the invention relating to wound care include methods for the treatment or prevention of infection and/or inhibition of bacterial colonization of a wound site. The latter method preferably utilizes a dressing, at least a portion of which overlays the wound site, and has incorporated therein the above-described antibacterial agent or composition. In either embodiment, the applied antibacterial composition may comprise an antibacterially-activated creatinine precursor or pharmaceutically acceptable salt thereof in an anhydrous carrier, with the water content of the integument in and around the wound site effecting conversion of the precursor to antibacterially-activated creatinine. Preferably, the composition is applied as a dry powder.

The antibacterial agents and compositions of the invention may additionally be used in a method of suppressing or preventing formation of body odor, due to odor-causing bacteria, by applying to at least one body part affected by body odor, e.g., the axilla or feet, an antibacterial composition as described herein. Here again, a combination of antibacterial-activated creatinine and an ester or other precursor thereof may be utilized to afford long-lasting protection against odor-causing bacteria.

Another method of the invention involves the treatment or prophylaxis of bacterial colonization of a bodily orifice of a subject, and tissue adjacent such orifice by delivering to the bodily orifice and/or adjacent tissue an antibacterial composition comprising an effective amount of at least one of antibacterially-activated creatinine, a pharmaceutically acceptable salt of antibacterially-activated creatinine, a precursor of antibacterially-activated creatinine, a pharmaceutically acceptable salt of the precursor and a dermatologically acceptable carrier medium. This method may be applied to treat or prevent bacterial colonization of a subject's nasal cavity, ear canal, lip, urethra, vagina or rectum. The preferred route of delivering the antibacterial agent in practicing the method is by spray, swab, drops or wash. In the case of treating or preventing bacterial colonization of the nasal cavity, the antibacterial composition is advantageously delivered by inhalation or by spraying, preferably in powder form. The antibacterial agent may be combined with a pharmaceutically acceptable bulking agent, and optionally an aerosol propellant in an amount sufficient to produce an aerosolized bolus containing the active agent.

In another method of using this invention, the antibacterial agent can be incorporated into a variety of substrates, thereby making them resistant to bacterial colonization. The substrate can be a fibrous material including, without limitation, cotton, nylon, rayon, polyester, polyurethane, wool or a combination thereof. The fibers may be made by conventional fiber-forming techniques, such as spinning or extrusion. The fibrous material may be in non-woven or woven form, examples of which are gauze and muslin. Other physical forms of substrates to which the method may be applied include cast or blown sheets and films, molded substrates and foam substrates, as well as paper, cardboard, pressed wood or fiber board materials.

In one embodiment, an aqueous solution of activated creatinine is applied, e.g., by padding, to sterile dry cloth and allowed to dry. As shown in the following examples, 100-200 mM aqueous solutions of creatinine are highly effective to inhibit growth and kill bacteria. Alternative modes of delivering the creatinine to the cloth or other fibrous materials include spraying, dipping (immersion) or bringing dry creatinine into contact with the substrate material. The antibacterial agents described herein may also be used to impart antibacterial properties to a wide range of polymer resins, including thermoplastic and thermosetting resins. Polymer resins are commonly used to provide a water-proof barrier to “soft” substrates, such as broadcloth, canvas, plastic sheet or film (e.g., tent liners), all-weather apparel, footwear and the like. For example, polyvinyl chloride (PVC), polyvinyl fluoride, polyurethane rubber and other resins used as water-proofing materials for lamination to, impregnation in, or coating on various substrates may be made bacterially resistant by incorporating therein an antibacterial agent of the invention. A coating composition could be formulated for durability, or could be reapplied at point of use in order to maintain antibacterial activity.

Other coating materials which can be rendered bacteria resistant are polymer-based paint systems used to coat rigid substrates, e.g., epoxy paints.

The antibacterial agent may be physically mixed or blended with a polymer resin laminating, coating or impregnating composition. Molded and foam articles made from polystyrene, polyurethane, polymethyl methacrylate and poly-ε-caprolactam can likewise be made resistant to bacteria in this way. Alternatively, due to the reactive nature of the antibacterial agents described herein, they may be covalently bound to a polymer laminating, coating or impregnating material, e.g., as a pendant group on a polymer backbone. Instead of incorporating the antibacterial agents of the invention into a substrate coating, laminating or impregnating composition, it may also be feasible to incorporate the agent into the substrate itself, via chemical binding to the substrate material. In the case of a polyester substrate, for example, one or more monomer units may be derivatized with the antibacterial agent of the invention. Additional polymers that may be chemically modified in this way include poly(ethylene-vinyl acetate), and polyamides/aramids, such as nylon, Kevlar® and Nomex

Similarly, molded articles of manufacturer can be engineered to contain antibacterially-activated creatinine, a pharmaceutically acceptable salt of said antibacterially-activated creatinine, a precursor of antibacterially-activated creatinine, a pharmaceutically acceptable salt of said precursor or any combination thereof. Furthermore, medical devices formed from injection molded plastic, such as medical catheters or endotraceal tubes, may be made using polymer compositions in which the antibacterial agent is physically or chemically incorporated.

While not wishing to be confined to any particular theory as to the mechanism of action of the above-described antibacterials, which has not been investigated, it is believed that the observed bacteriostatic/bacteriocidal effect is due to interference with one or more of the three arginine biosynthesis pathways, feeding back and halting arginine synthesis, which in turn halts bacterial cell replication. The antibacterials of the invention could also alter the charge of the bacterial cell wall or obstruct its ion channels through interactions with the activated molecule and its counterion, leading to disruption of the cell wall. Yet another possibility for the mechanism of action of the antibacterials of the invention is by influencing the activation, either positively or negatively, of the autolytic regulatory genes, Arg and Sar, either by direct action on the promoter or indirectly by creating alterations in the citric acid or acetate metabolic pathways, as described for Triton X-100 and Penicillin-induced autolysis. Fujimoto and Bayles, J. Bacteriol. 180: 3724-3726 (1998).

Another practical application of the antibacterial agents of this invention involves their use in a method of suppressing bacterial growth in a culture comprising a eukaryotic organism and a growth medium for such organism, by incorporating in the growth medium an antibacterially effective amount of one or more of the antibacterials described herein. This method can improve the commercial production of fungi, such as Baker's yeast or Brewer's yeast. It can also be applied in drug discovery and development, by enabling the isolation and identification of pure cultures of infectious agents from the group of invasive candidiasis, invasive aspergillosis, zygomycosis, disseminated cryptococcosis, disseminated histoplasmosis, and trichosporon species.

Experiments performed to date have shown that the antibacterial agents of the invention have good thermal stability (from −10° C. to 45° C.) and long shelf-life (anhydrous cream formulation was fully active after two (2) years of storage).

In testing the above-described antibacterial agents, no resistance was seen to develop in bacteria passed multiple times in media containing sub-bacteriostatic concentrations of antibacterially-active creatinine, and then plated on agar that contains bacteriostatic concentrations thereof.

The following examples describe the invention in further detail, with reference to specific embodiments. These are representative embodiments of the invention which are provided for illustrative purposes only, and which should not be regarded as limiting the invention in any way.

### Example 1

A 2 molar solution of anhydrous creatinine (Sigma-Aldrich Chemical Co. St. Louis, Mo.) was prepared in water (113 mg in 0.5 ml H₂O) and adjusted to pH 5.0-5.5 with different acids. Twenty five microliters of each pH adjusted solution was added to 30 milligrams of a powdered carrier, Eridex™, (crystalline sugar alcohol) (Cargill Inc. Cedar Rapids, Iowa), and stirred into a thickened slurry. Approximately 50 microliters of each mixture, containing 5 mg of acidified creatinine, was applied to a 6 mm disc and inverted onto a brain heart infusion agar plate that was spread one hour prior with Staphylococcus aureus diluted to 10⁵ organisms per milliliter. Plates were incubated at 37° C. overnight and the clear zones showing inhibition of bacterial growth were measured. Each sample was run in duplicate and reported as an average +/−1 mm of the two measurements. Creatinine HCl was used as an internal standard on each plate to provide uniformity from plate to plate (measurements varied <0.5 mm). Hydrochloride sig, a commercial creatinine HCl salt from Sigma-Aldrich, was used at pH 5.0 with no adjustment.

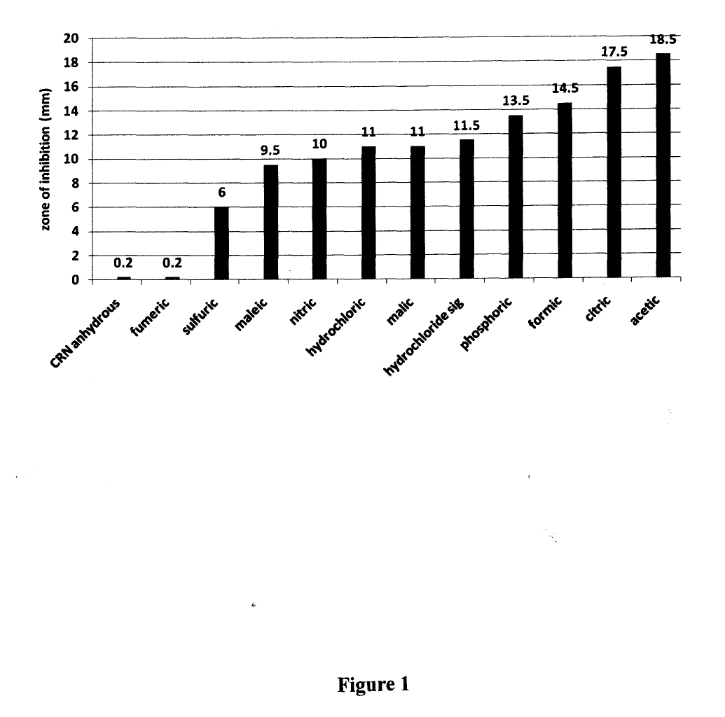

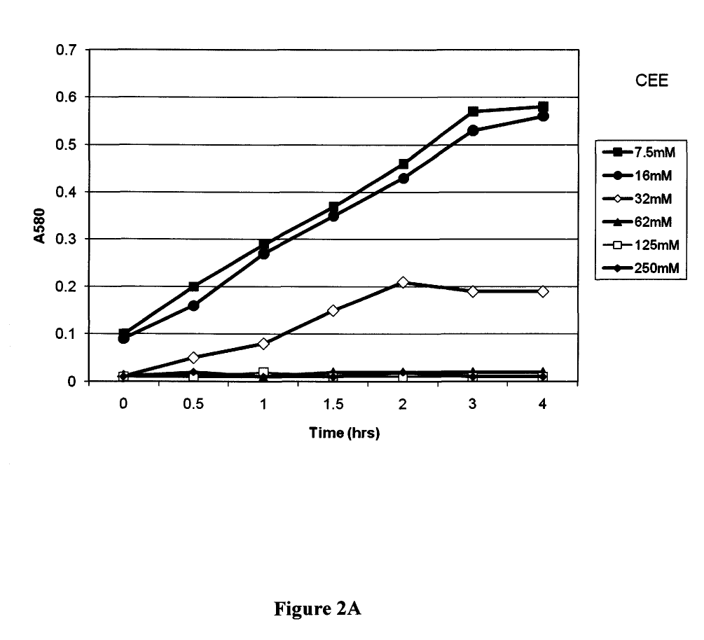

The results are shown in FIG. 1, in which it can be seen that the degree of activation depends on the acid used to adjust the pH, since the different levels of antibacterial activity were found to vary depending upon the salt formed or the counterion associated with the creatinine in the resulting solution. As shown in FIG. 1, creatinine salts/counterions resulting from treatment with sulfuric or fumaric acid have little to no antibacterial activity, whereas acetate and citrate salts/counterions demonstrate significant activity. Additionally, the controls of acetic acid or citric acid in Eridex™ alone, without creatinine at pH 5.5, demonstrate no antibacterial activity.

### Example 2

Compositions including the antibacterial agents described herein can be made by formulation procedures commonly used in the pharmaceutical and cosmetics industry.

For purposes of the experiments described below, test compositions were prepared by admixing antibacterially-activated creatinine (CRN) or a precursor of antibacterially-activated CRN, i.e., creatine ethyl ester (CEE), as required for the experiment at hand, with a weighed amount of a suitable carrier medium to give a final concentration of 28% by weight of the antibacterial agent, based on the total weight of the composition.

Antibacterial compositions were prepared following this procedure using a number of different aqueous and non-aqueous (anhydrous) carrier media, and evaluated for antibacterial activity in a standard disc diffusion assay. Thus, approximately 50 μL of each selected carrier containing 28% by weight of either a precursor of antibacterially-activated CRN, CEE, or antibacterially-activated CRN was added to a 6 mm disc and inverted onto a brain heart infusion agar plate that was previously spread with S. aureus at 10⁵ organisms per mL. Plates were incubated at 37° C. overnight and the clear zones showing inhibition of bacterial growth were measured.

The recorded measurements are listed in Table 1, below, and show that both antibacterially-activated CRN and its precursor, CEE, possess similar activity in a standard disc assay.

TABLE 1

Zone of Inhibition
(mm)

| | CEE | CRN |
|---|---|---|
| Aqueous Carrier | | |
| water | 9 | |
| water/25% glycerol | 15 | nd^{a} |
| water/50% glycerol | 15 | nd |
| hydrogel | 20 | 22 |
| Anhydrous Carrier | | |
| mineral oil | 21 | 25 |
| polyethylene glycol 200 | 18 | nd |
| polyethylene glycol 400 | 14 | 16 |
| red palm oil | 15 | nd |
| white palm oil | 12 | nd |
| white palm oil/corn oil (50% ea) | 17 | nd |
| pharmaceutical grade Lipoil^{b} | 12 | 16 |
| pharmaceutical topical formulation^{c} | 26 | 27 |

^{a}not done
^{b}mixture of lecithin and isopropyl palmitate
^{c}proprietary blend of lower monohydric alcohols, higher monohydric alcohols, diols, diol-monoesters and fatty acids.

### Example 3

An experiment was conducted with a precursor of antibacterially-activated CRN, i.e., CEE, as the antibacterial agent and tested on S. aureus and M. luteus plates for antibacterial activity.

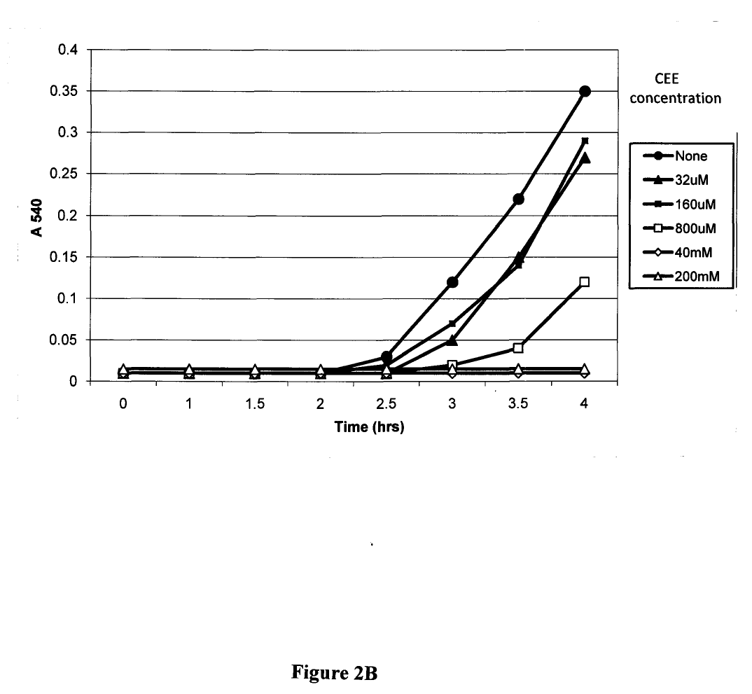

The CEE precursor of antibacterially-activated CRN was diluted at different concentrations into Lederberg's broth (LB), which was then inoculated to 10⁴/mL with S. aureus. The cultures were incubated in capped plastic tubes at 37° C. and aerated by tumbling using a tube rotating device. Absorbance readings were plotted as a function of time, and the results are set forth in FIG. 2A. The data show that at a concentration of approximately 32 mM or greater, the CEE precursor of antibacterially-activated CRN inhibited growth of S. aureus.

A similar experiment was conducted with the same precursor of antibacterially-activated CRN and tested on M. luteus. Absorbance readings were plotted as a function of time and the results are set forth in FIG. 2B. The data show that at a concentration of approximately 1 mM or greater, the CEE precursor of antibacterially-activated CRN, inhibited growth of M. luteus.

FIGS. 2A and 2B also show the different levels of sensitivity of two different organisms to the antibacterially-activated CRN. S. aureus required a concentration of 32 mM for inhibition of growth, whereas as little as 1 mM concentration inhibited the growth of M. luteus.

### Example 4

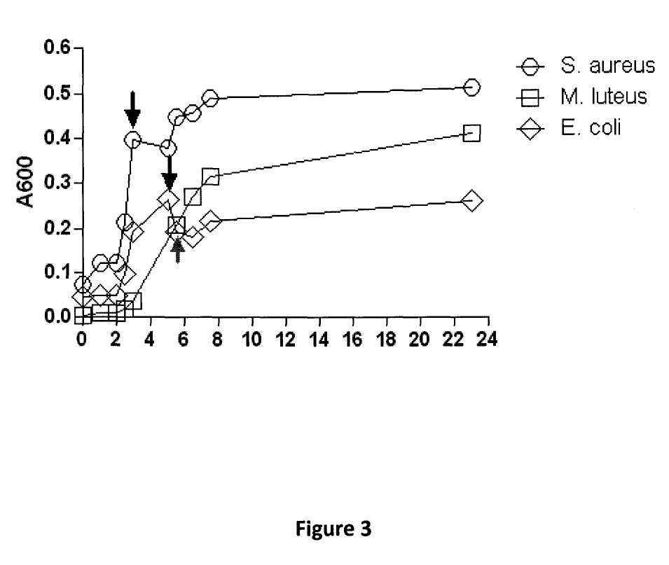

A 0.5 mL aliquot of an overnight culture of each of three (3) bacterial strains (i.e., S. aureus, M. luteus and E. coli) was added to 50 mL LB and shaken at 225 RPM at 37° C. Absorbance readings were made, and when cells were in early log phase, CRN from a 2M sterile stock solution was added to the culture to a final concentration of 100 mM (arrows indicate addition of CRN). Absorbance readings were plotted as a function of time, over a 24 hour period, and the results are set forth in FIG. 3. The data show that CRN halted bacterial growth even when actively growing bacteria, in mid-log phase.

### Example 5

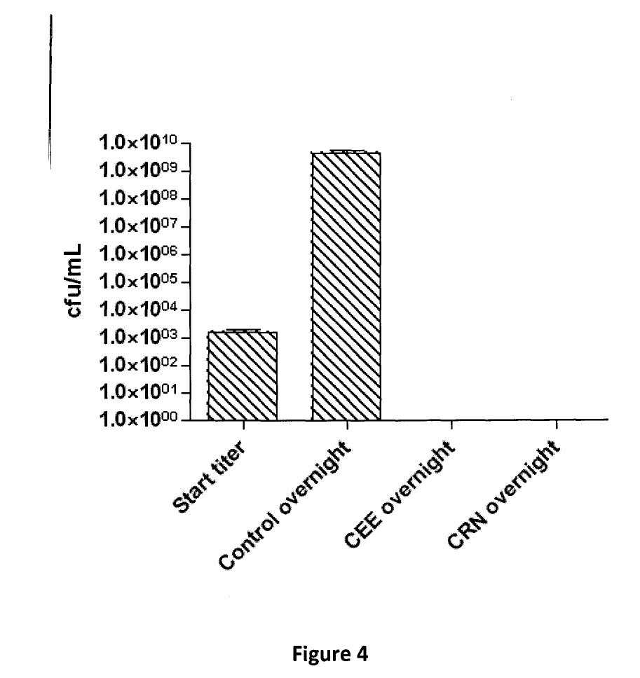

S. aureus UAMS-1 was started from a static overnight, grown for 3 hours, then 0.2 mL was added to 3 different 50 mL cultures for control, CRN and CEE. These were shaken 30 min. at 37° C., an aliquot was removed prior to the addition of a precursor of antibacterially-activated CRN, CEE, or antibacterially-activated CRN, and the starting titer was determined. Powdered CEE or crystal CRN was added directly to the 50 mL cultures to a final concentration of 200 mM. The cultures were shaken overnight and the absorbance (A) and colony forming units (cfu) were determined. The cultures treated with the antibacterial agents were clear and 1 mL of the culture plated on solid media demonstrated no colonies after 48 hours incubation, while the control plated out to 3×10⁹ per mL The results are shown in FIG. 4.

This experiment demonstrated that antibacterially-activated CRN and a precursor of antibacterially-activated CRN, CEE, are both bacteriocidal at 200 mM for S. aureus when starting at about 1×10³ organisms per mL.

### Example 6

Four (4) different creatine esters were prepared according to the method of U.S. Pat. No. 6,897,334 to Vennerstrom. The esters thus prepared were activated in the manner described herein to impart antibacterial activity thereto. Four (4) test formulations were prepared, each including approximately 50 μL of an anhydrous polyethylene glycol carrier containing 28% by weight of a different creatine ester. Each formulation was added to a 6 mm disc and inverted onto a brain heart infusion agar plate that was previously spread with S. aureus at 10⁵ organisms per mL. Plates were incubated at 37° C. overnight and the clear zones showing inhibition of bacterial growth were measured.

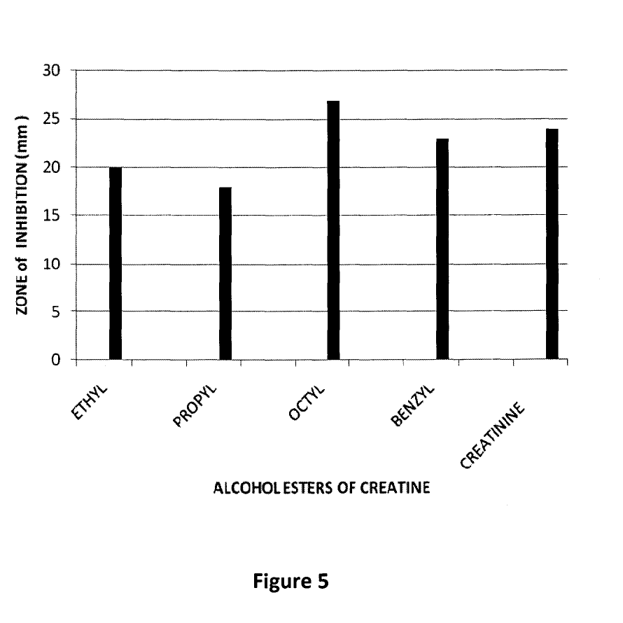

As can be seen in Table 2 below and in FIG. 5, four (4) different formulations of creatine ester demonstrated antibacterial activity against Staphylococcus that was similar to that of antibacterially-activated CRN.

TABLE 2

| Creatine Ester | Zone of Inhibition (mm) |
|---|---|
| Ethyl | 20 |
| Propyl | 18 |
| Octyl | 27 |
| Benzyl | 23 |
| Creatinine (no ester) | 24 |

### Example 7

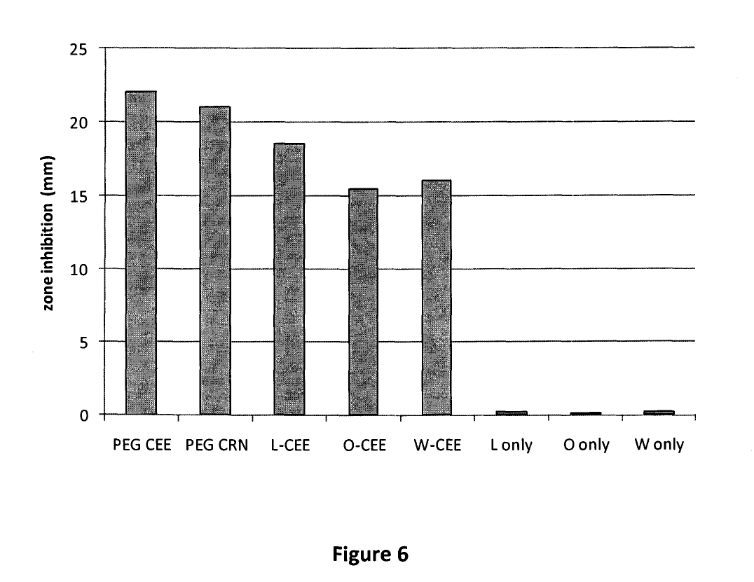

A precursor of antibacterially-activated CRN, i.e., CEE, was admixed with three (3) different commercially available water-based lotions Lubriderm (L), an organic based generic lotion (O) and a Walgreens skin lotion (W) and tested for antibacterial activity in a standard disc diffusion assay. The antibacterial activity of these lotions supplemented with CEE was compared to anhydrous formulations of either CEE or CRN based on pharmaceutical grade polyethylene glycol and Lipoil (PEG-CEE and PEG-CRN respectively. Approximately 50 μL of each carrier containing either a precursor of antibacterially-activated CRN, CEE, or antibacterially-activatedCRN was added to a 6 mm disc and inverted onto a brain heart infusion agar plate that was previously swabbed with S. aureus at 10⁵ organisms per mL. Plates were incubated at 37° C. overnight and the clear zones showing inhibition of bacterial growth were measured. The concentration of a precursor of antibacterially-activated CRN,CEE, and antibacterially-activated CRN in the formulations tested was 500 mM or approximately 10% by weight. The results of this experiment, which are set forth in Table 3 below and in FIG. 6, demonstrate that a precursor of antibacterially-activated CRN, CEE, may enhance existing commercial skin care products by supplementing their intrinsic properties with antibacterial or antiseptic activity.

TABLE 3

| | Zone of Inhibition (mm) |
|---|---|
| PEG CEE | 22 |
| PEG CRN | 21 |
| L-CEE | 18.5 |
| O-CEE | 15.5 |
| W-CEE | 16 |
| L only | 0.2 |
| O only | 0.1 |
| W only | 0.2 |

### Example 8

Separate formulations of antibacterially-activated CRN and a precursor of antibacterially-activated CRN, CEE, were prepared by admixing the respective antibacterial agent with a hydrogel (Advanced Medical Solutions Ltd, UK.) in varying amounts to provide final concentrations of 1 M, 500 mM, 250 mM and 125 mM. Approximately 50 μL of each formulation at the four (4) different molar concentrations was added to a 6 mm disc and inverted onto a brain heart infusion agar plate that was previously spread with S. aureus suspended in PBS at 10⁵ organisms per mL. Plates were incubated at 37° C. overnight and clear zones showing inhibition of bacterial growth were measured. The hydrogel carrier including no bacterial agent (0 mM) was used as a control.

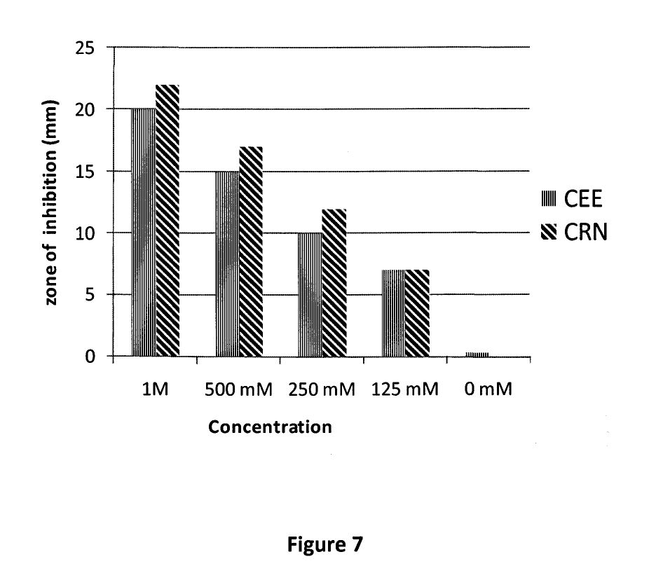

The data obtained from this experiment are presented in Table 4, below, as well as in FIG. 7.

TABLE 4

Zone of Inhibition
(mm)

| | CEE | CRN |
|---|---|---|
| 1M | 20 | 22 |
| 500 mM | 15 | 17 |
| 250 mM | 10 | 12 |
| 125 mM | 7 | 7 |
| 0 mM | 0.3 | 0 |

### Example 9

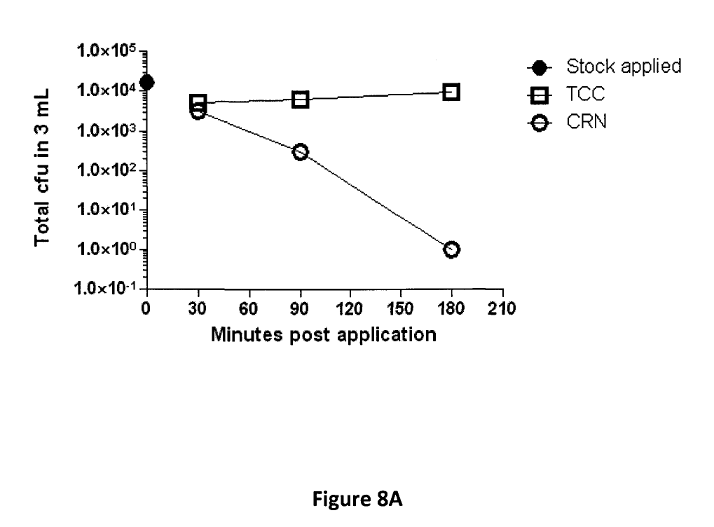

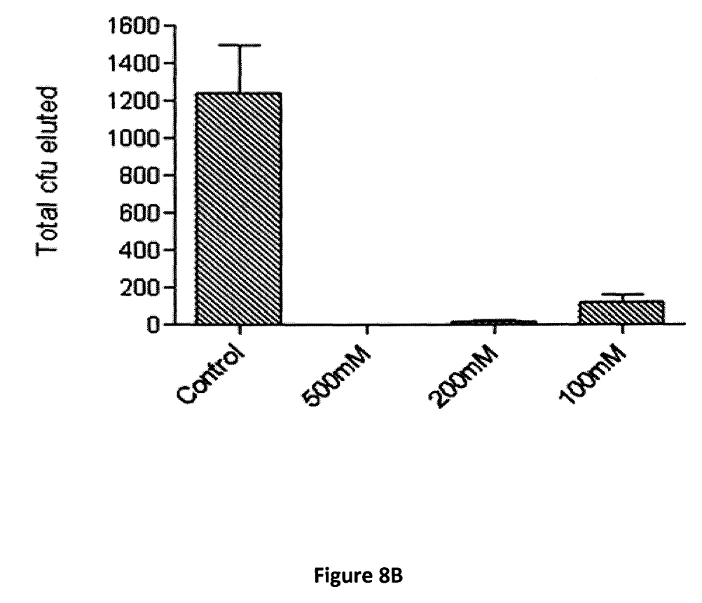

To test if CRN inhibited bacterial growth on cloth or potential wound dressing, the agent was applied to 1 cm² pieces of sterile, dry lab coat cloth (65% polyester, 35% cotton), to which bacteria were then applied. A control cloth was used which was not treated. After specific times the cloth was suspended in media and vortexed to dislodge the bacteria. An aliquot of the bacterial suspension thus obtained was plated and the resulting colony-forming units (cfu) were determined. Next, 25 μL of 2M CRN was spotted onto the cloth, then dried at 37° C. overnight in a sterile petri dish. 25 uL of a log phase culture of S. aureus (=1-2×10⁵ cfu) was spotted on the cloth samples and placed spot side up in a sterile petri dish at 37° C. At selected times following incubation, the cloth samples were placed into 3 mL of LB and vortexed repeatedly for 5 minutes. One mL (33%) of the total volume of bacterial suspension was plated to determine the cfu/mL. The results are presented in FIG. 8A. No cfu could be detected from the CRN-treated cloth after 3 hours, compared to the non-treated, culture control cloth (CC) that maintained a constant number of organisms (approximately 10⁴ cfu). The results show that CRN was bacteriocidal under these conditions within 3 hours of bacterial application.

The effects of different concentrations of antibacterially-activated CRN on bacterial growth was determined by applying the antibacterial agent at various concentrations to the cloth pieces. Different cloth samples were treated by spotting 25 uL of 500 mMm 200 mM or 100 mM antibacterially-activated CRN to the material. The samples were maintained overnight at 37° C. and then processed in the manner described immediately above. The results obtained are shown in FIG. 8B.

Bacterial cfu were decreased with all three (3) concentrations tested. No S. aureus cfu were recovered from the cloth treated with 500 mM antibacterial agent. Moreover, a significant reduction in cfu was demonstrated at both the 200 and 100 mM concentrations tested, thus indicating that treating cloth or wound dressing material within these ranges of concentrations of antibacterially-activated CRN would be an effective and efficient means of inhibiting bacterial growth and potentially reducing wound infections.

### Example 10

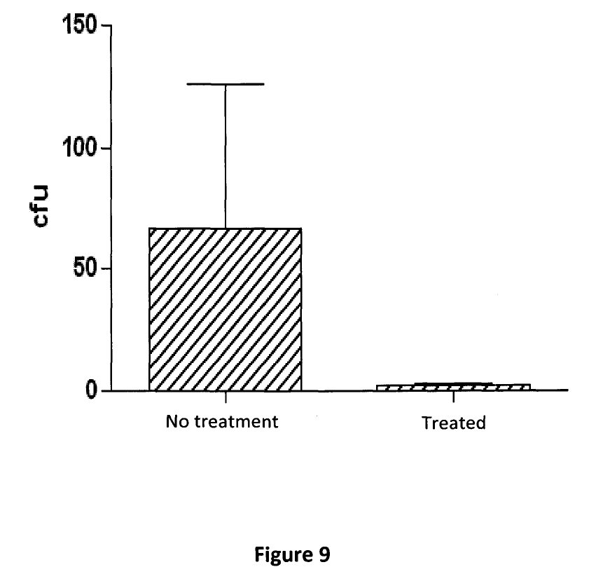

The index finger of each hand was pressed onto one quadrant of a Brain Heart Infusion (BHI) agar plate in order to estimate the background count of the normal bacterial flora. The plate was set at room temperature. Each finger was then covered with a generic brand commercial sterile bandage (Walgreens) that was pretreated experimentally with CEE formulated in a polyethylene glycol 400 (Gallipot, St Paul Minn.) base to 28% by weight. After 4 hours, the bandages were removed and each finger blotted onto the remaining two quadrants of the plate. The plate was incubated at 37° C. and the number of cfu determined. The experiment was repeated three (3) times on three (3) different days and the results are shown in FIG. 9.

When dressing/Band-Aid or other bandage containing a precursor of antibacterially-activated CRN, CEE, is applied to a wound in the “normal” warm, moist environment of the wound, the bacteria that are present would be killed when they are actually growing. Thus, the bacteria would not adhere to, colonize or infect the wound.

### Example 11

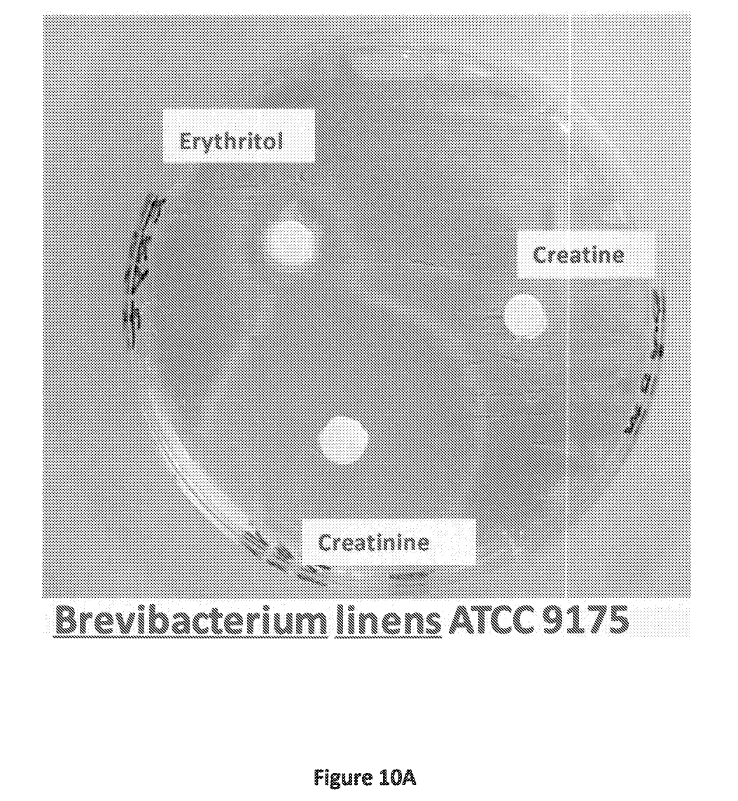

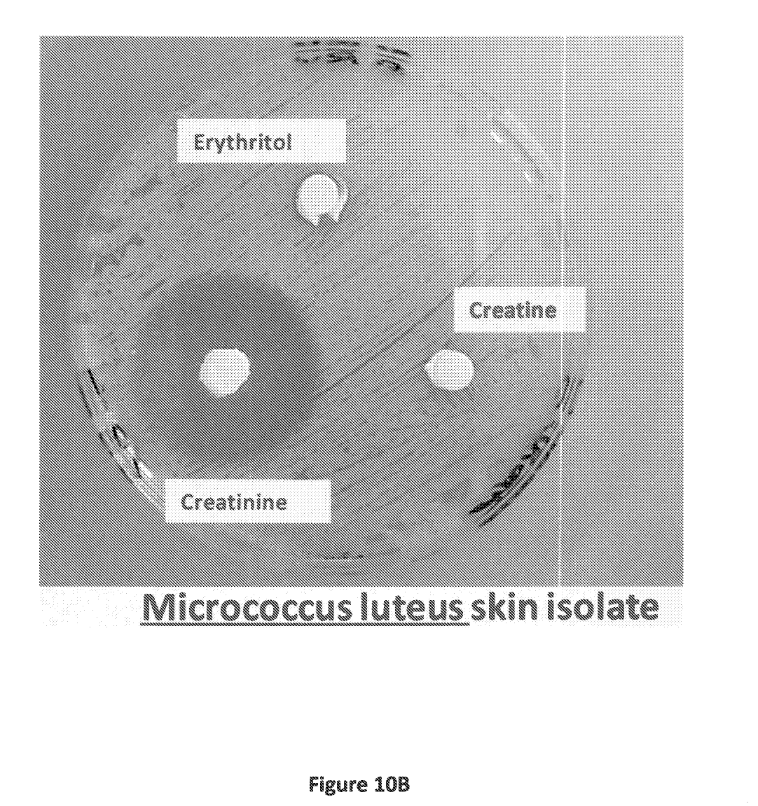

Twenty-five milligrams of an anhydrous formulation of either antibacterially-activated CRN or a precursor of antibacterially-activated CRN, CEE, was prepared as described in Example 2, applied to a filter disc and tested for inhibition of bacterial growth of several odor causing microorganisms in a standard disc diffusion assay using Eridex™ (Cargill Inc. Cedar Rapids, Iowa) or creatine monohydrate containing no antibacterial agent as controls. Effective inhibition of growth was observed for all odor causing organisms tested. As can be seen in Table 5 below, zones of inhibition ranged from 43 mm with Brevibacterium linens to 20 mm with Bacillus subtilis. The zones of inhibition for CRN or lack thereof for control test samples for B. linens and M. luteus are illustrated in FIGS. 10A and 10B respectively.

TABLE 5

ZONE OF INHIBITION (mm)

| ORGANISM | CRN | CEE |
|---|---|---|
| Brevibacterium linens | 43 | 40 |
| Micrococcus luteus | 20 | 22 |
| Bacillus subtilis | 24 | 20 |
| Staphylococcus epidermidis | 27 | 27 |

### Example 12

The antibacterially-activated CRN or a precursor of antibacterially-activated CRN, i.e., CEE, was tested for possible use in generating a selective growth environment for yeast and other fungi.

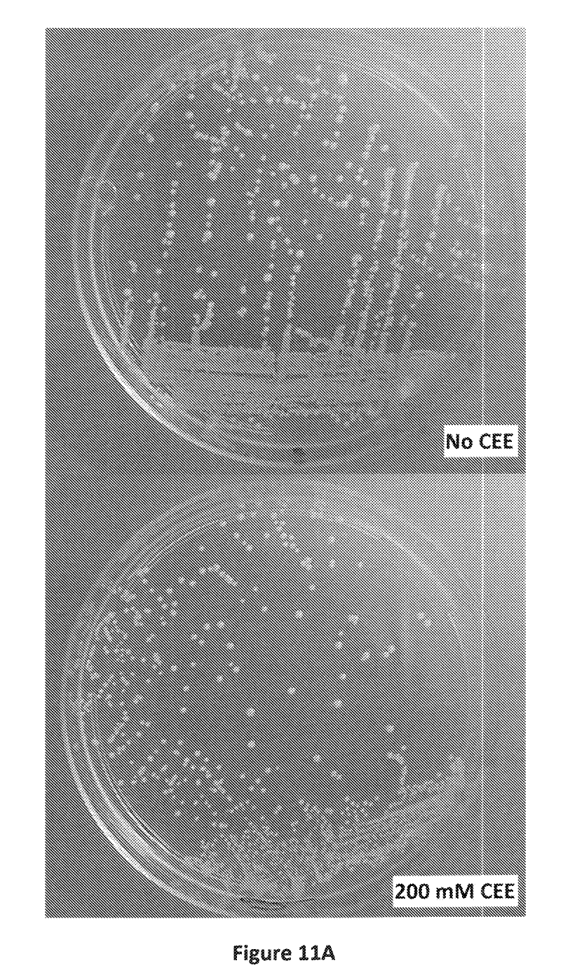

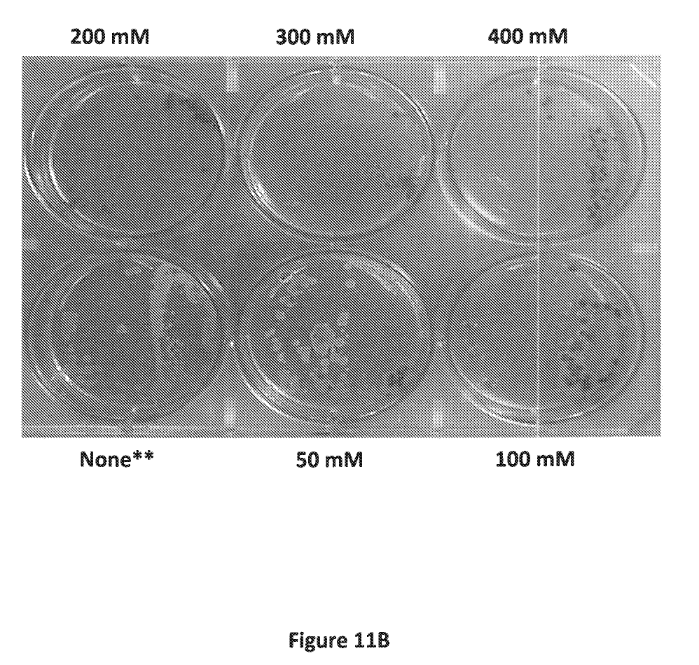

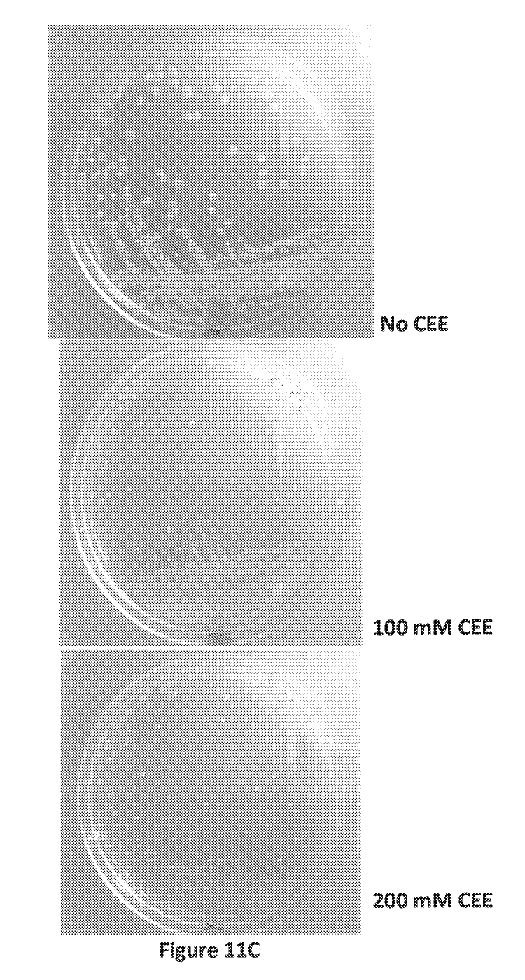

Colonies of Micrococcus sp. and Saccharomyces sp. grown on LB agar supplemented with 1% dextrose (LBD) were suspended in PBS to 0.30 A₅₈₀ nm. An equal volume of the two organisms was combined and the mixture streaked onto LBD agar alone or LBD containing CEE and incubated at 37 C for 24 hours. The media was selective for Saccharomyces at 200 mM CEE as shown in FIG. 11A. CEE concentrations from 300-400 mM CEE also supported growth of Saccharomyces (data not shown).

Colonies of S. aureus and Rhodotorula sp. grown on LB agar supplemented with 1% dextrose (LBD) were suspended in PBS to 0.30 A₅₈₀ nm. An equal volume of the two organisms was combined and the mixture streaked onto wells containing only LBD (None) or LBD supplemented with a precursor of antibacterially-activated CRN, CEE, and incubated at 37° C. for 36 hours. As shown in FIG. 11B, Growth of Staphylococcus was retarded at 100 mM CEE and completely inhibited at 200 mM and greater. Concentrations of 200-400 mM CEE did not retard growth of Rhodotorulia

Colonies of Micrococcus sp., Rhodotorula sp. and Saccharomyces sp. grown on brain heart infusion agar (BHI) were suspended in PBS to 0.30 OD, 580 nm. An equal volume of the three (3) organisms were combined and the mixture streaked onto plates containing BHI agar alone or BHI agar supplemented with a precursor of antibacterially-activated CRN, CEE, and incubated 48 hours at 37° C. As shown in FIG. 11C, growth of Micrococcus was greatly retarded at 100 mM CEE and completely inhibited at 200 mM whereas the growth of both yeast species was enhanced at both concentrations. CEE at 300 and 400 mM also inhibited the growth of Micrococcus and did not affect the growth of either Rhodotorula or Saccharomyces (data not shown).

In summary, as those skilled in the art will appreciate upon reading the foregoing description, the present invention provides bacteriostatic agents of general utility, which also exhibit bactericidal action against actively growing bacteria, and which may be applied anywhere topical antibiotics are currently in use, either as a replacement for or adjunct to existing antibiotics. By arresting bacterial reproduction, the antibacterial agents of this invention may inhibit the development of multiple toxic and defense systems, thus rendering the bacteria more susceptible to antibiotics and natural host defenses.

A number of patent documents and non-patent documents are cited in the foregoing specification in order to describe the state of the art to which this invention pertains. The entire disclosure of each of the cited documents is incorporated by reference herein.

It should be noted that, as used in the preceding description and the appended claims, the singular articles “a”, “an” and “the” also include the plural, unless the context clearly indicates otherwise.

While various embodiments of the present invention have been described and/or exemplified above, numerous other embodiments will be apparent to those skilled in the art upon review of the foregoing disclosure. The present invention is, therefore, not limited to the particular embodiments described and/or exemplified, but is capable of considerable variation and modification without departure from the scope of the appended claims. Furthermore, the transitional terms “comprising”, “consisting essentially of” and “consisting of”, when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All of the antibacterial agents, compositions and products containing such agents and the methods of use thereof which embody the present invention can, in alternate embodiments, be more specifically defined by any of the transitional terms “comprising”, “consisting essentially of” and “consisting of”.

1. An antibacterial composition comprising, as the active agent, antibacterially-activated creatinine, a pharmaceutically acceptable salt of said antibacterially-activated creatinine, a precursor of antibacterially-activated creatinine, a pharmaceutically acceptable salt of said precursor, or a combination thereof, and a carrier medium.

2. The composition of claim 1, wherein said precursor of antibacterially-activated creatinine is at least one of creatine, a pharmaceutically acceptable salt of creatine, a creatine ester, or a pharmaceutically acceptable salt of a creatine ester.

3. The composition of claim 1, wherein said active agent is a precursor of antibacterially-activated creatinine, said precursor being a creatine ester or pharmaceutically acceptable salt of a creatine ester.

4. The composition of claim 1, wherein said active agent is the precursor of antibacterially-activated creatinine, creatine ethyl ester, or a pharmaceutically acceptable salt of said ester.

5. The composition of claim 1, wherein said active agent is present in said composition at a concentration of at least 10 mM.

6. The composition of claim 1, wherein said active agent is present in said composition at a concentration in the range from about 100 mM to about 2 M.

7. The composition of claim 1, wherein said active agent is present in said composition in an amount of at least 0.5%, based on the total weight of the composition.

8. The composition of claim 1, wherein said active agent is present in said composition in an amount from about 3% to about 99.5%, based on the total weight of the composition.

9. The composition of claim 1, comprising a combination of antibacterially-activated creatinine and a precursor of antibacterially-activated creatinine.

10. The composition of claim 1, wherein said carrier medium is an anhydrous medium selected from the group consisting of mineral oil, polyethylene glycol, vegetable oil, fatty acid, propylene glycol, glycerin, alcohol, parafin, or a mixture thereof.

11. The composition of claim 10 in the form of a cream, gel, ointment, paste, suspension or spray.

12. The composition of claim 1, wherein said composition comprises at least one additional ingredient selected from the group consisting of emollients, humectants, surfactants (such as sugar esters, lanolin oils, waxes); cosmetic modifiers and emulsifiers.

13. The composition of claim 1, wherein said carrier is an aqueous medium selected from the group consisting of water and hydroalcoholic solutions.

14. The composition of claim 13 in the form of a cream, lotion, gel, suspension, emulsion, or spray.

15. The composition of claim 1 in powder form, and further comprising a pharmaceutically acceptable bulking agent, and, optionally, an aerosol propellant in an amount sufficient to produce an aerosolized bolus containing said active agent.

16. The composition of claim 1 comprising an anti-infective agent.

17. The composition of claim 16, wherein said anti-infective agent is selected from the group consisting of an antibiotic, antifungal, antiseptic, and antiviral agent.

18. A wound dressing comprising a wound dressing material in which is incorporated an antibacterially effective amount of at least one of antibacterially-activated creatinine, a pharmaceutically acceptable salt of said antibacterially-activated creatinine, a precursor of antibacterially-activated creatinine, a pharmaceutically acceptable salt of said precursor.

19. The wound dressing of claim 18, wherein said wound dressing material is selected from the group of a hydrocolloid, a hydrogel, a semi-permeable transparent film, an open-cell foam, an alginate, an absorptive filler, a woven fabric and a non-woven fabric, or a combination of said materials.

20. The wound dressing of claim 18, which is a hydrocolloid comprising an absorbent and elastomer dispersed in an adhesive base.

21. The wound dressing of claim 18 comprising a combination of antibacterially-activated creatinine and a precursor of antibacterially-activated creatinine.

22. The wound dressing of claim 20 further including a transparent cover film for securing said wound dressing to a wound site, said cover film being impermeable to liquid, bacteria and viruses.

23. The wound dressing of claim 20, wherein said hydrocolloid is in wafer, powder or paste form.

24. The wound dressing of claim 18, which is a hydrogel comprising cross-linked hydrophilic macromolecules containing up to about 95% water, by weight.

25. The wound dressing of claim 24 comprising a combination of antibacterially-activated creatinine and a precursor of antibacterially-activated creatinine.

26. The wound dressing of claim 24, wherein said hydrogel is in sheet or gel form.

27. The wound dressing of claim 26 in gel form, said gel being impregnated in gauze.

28. The wound dressing of claim 18, wherein said wound dressing material comprises a bandage strip and an absorbent compress attached to said bandage strip.

29. The wound dressing of claim 28, which is a dry dressing or a water dressing.

30. The wound dressing of claim 29, wherein said dressing comprises cotton, nylon, polyester, polyurethane, wool or a combination thereof.

31. The wound dressing of claim 28, wherein said absorbent compress is an open-cell foam material.

32. The wound dressing of claim 28, wherein said absorbent compress is gauze.

33. The wound dressing of claim 28 contained in an air-tight container.

34.-50. (canceled)

51. A method of inhibiting growth of bacteria, said method comprising localized administration to a region in need of bacterial growth inhibition an antibacterially effective amount of at least one of antibacterially-activated creatinine, a pharmaceutically acceptable salt of said antibacterially-activated creatinine, a precursor of antibacterially-activated creatinine, a pharmaceutically acceptable salt of said precursor.

52. The method of claim 51, wherein a combination of antibacterially-activated creatinine and a precursor of antibacterially-activated creatinine is administered.

53. The method of claim 51, wherein said composition is administered for inhibiting the growth of at least one organism selected from the group consisting of Staphylococcus aureus, Enterococcus faecalis, Pseudomonas aeruginosa, Pseudomonas fluorescens, Escherichia coli, Acinetobacter baumannii, Brevibacterium linens, Micrococcus luteus, Bacillus subtilis, Bacillus cereus.

54. The method of claim 51, wherein said composition is administered for inhibiting the growth of at least one antibiotic resistant organism.

55. The method of claim 54, wherein said antibiotic resistant organism is selected from the group consisting of methicillin-resistant S. aureus (MRSA), Acinetobacter baumannii high level resistance, E. coli beta lactamase producer, vancomycin-resistant Enterococci (VRE), and Pseudomonas aeruginosa high level resistance

56. The method of claim 51, wherein said composition is administered to an animal.

57.-60. (canceled)

61. The method of claim 56, wherein said composition is topically administered.

62.-68. (canceled)

69. A method of inhibiting bacterial colonization of a wound site, said method comprising applying a dressing to said wound site, said dressing having at least a portion overlaying said wound site, said portion having incorporated therein an antibacterially effective amount of at least one of antibacterially-activated creatinine, a pharmaceutically acceptable salt of said antibacterially-activated creatinine, a precursor of antibacterially-activated creatinine, a pharmaceutically acceptable salt of said precursor.

70. A method for the treatment or prophylaxis of infection in a wound, the method comprising applying to the wound site an antibacterial composition having as an active agent an effective amount of at least one of antibacterially-activated creatinine, a pharmaceutically acceptable salt of said antibacterially-activated creatinine, a precursor of antibacterially-activated creatinine, a pharmaceutically acceptable salt of said precursor.

71. The method of claim 70, wherein the applied antibacterial composition comprises an antibacterially-activated creatinine precursor or a pharmaceutically acceptable salt of said precursor in an anhydrous base, and water content of said wound site effects conversion of said precursor to antibacterially-activated creatinine.

72. The method of claim 70, wherein said composition is applied as a dry powder.

73. A method for making a substrate resistant to bacterial colonization, said method comprising incorporating into said substrate an antibacterially effective amount of at least one of antibacterially-activated creatinine, a pharmaceutically acceptable salt of said antibacterially-activated creatinine, a precursor of antibacterially-activated creatinine, a pharmaceutically acceptable salt of said precursor.

74. The method of claim 73, wherein a combination of antibacterially-activated creatinine and a precursor of antibacterially-activated creatinine is administered.

75. The method of claim 73, wherein said at least one antibacterially-activated creatinine, antibacterially-activated creatinine salt, antibacterially-activated creatinine precursor or antibacterially-activated creatinine precursor salt is incorporated into said substrate by coating, dipping or chemical binding.

76.-94. (canceled)
